(19) United States
(12) Patent Application Publication
(10) Pub. No.: US 2006/0169291 A1
(43) Pub. Date: Aug. 3, 2006

(54) Earplug

(76) Inventor: Yuichiro Shirai, Tokyo (JP)

Correspondence Address: HOWSON AND HOWSON, SUITE 210, 501 OFFICE CENTER DRIVE, FT WASHINGTON, PA, 19034, US

(21) Appl. No.: 11/050,213

(22) Filed: Feb. 3, 2005

(51) Int. Cl. A61F 11/00

(52) U.S. Cl. 128/864; 128/866

(57) ABSTRACT

An earplug comprises a plug body composed a material such as silicone, petroleum jelly, or cotton impregnated with petroleum jelly, which exhibits plasticity and can be deformed freely at ordinary temperatures. A plastic film cover enclosed the plug body. The plug body is in the shape of a bullet, with a tapered end adapted to point toward and enter the user's ear canal. The end of the cover at the end of the plug opposite to the tapered end is formed into a removal extension, which can be grasped manually for removal of the earplug.

### FIELD OF THE INVENTION

This invention relates to earplugs, and particularly to an improved earplug which can be removed easily, which can be used for a long time, and which exhibits a high degree of sound attenuation.

### BACKGROUND OF THE INVENTION

Conventional earplugs can be roughly classified into two groups, namely, sponge-type earplugs and pliable silicone earplugs.

A sponge-type earplug 10A is shown in FIGS. 4(a) and 4(b). This earplug is inserted by deforming it into a narrow roll and then pushing it into one's ear canal. The earplug 10A is resilient, soon recovers its form after being pushed into the ear canal, and adheres to the wall of the ear canal in order to produce a soundproofing effect. Because it is resilient, and expends inside the ear canal, the sponge-type earplug gives the user an oppressive sensation, and consequently cannot be kept in the ear comfortably for a long period of time.

Earplugs comprising silicone are widely used in the United States. As shown in FIG. 5, an earplug 10B, comprising silicone, can produce a soundproofing effect as well as a waterproofing effect. In use, it is initially nearly spherical. When used, it is placed over the outer opening of the ear canal and deformed by pressing with a finger so that it covers the part of the ear surrounding the outer opening.

The sponge-type earplug shown in FIGS. 4(a) and 4(b) does not attract ear wax. However, it is deficient in its ability to attenuate sound. It also loses its elastic properties when used repeatedly, and fails to function effectively as an earplug after a relatively short time.

The function of the silicone earplug shown in FIGS. 5(a) and 5(b) depends on the ability of the silicone to adhere to the user's skin. Because of its adhesive property, the silicone material attracts ear wax, and tends to become filthy. The silicone material also loses its adhesive property after a short time. Furthermore, when properly used, the silicone earplug is placed over the entrance of the ear canal rather than inserted. When the silicone earplug is inserted into the ear canal, it can be difficult to remove. And, in a worse case, if the earplug is broken, a piece of silicone material may be left inside the ear canal, requiring medical attention for its removal. Since the silicone earplug, when properly used, is placed over the entrance of the ear canal and relies upon the adherence of the silicone to the portion of the user's ear surrounding the entrance, the desired soundproofing effect is not necessarily achieved satisfactorily.

### SUMMARY OF THE INVENTION

Objects of this invention are to avoid one or more of the above-mentioned problems, and particularly to provide an earplug which is easily removable, which provides effective and reliable sound attenuation, and which may be used for a long time.

The earplug according to the invention comprises a material that exhibits sufficient plasticity at ordinary ambient temperatures that it cen be deformed manually, and a flexible film cover enclosing the material. A portion of the flexible film cover extends away from the enclosed material, forming an extension that can be grasped manually for removal of the earplug from the ear.

The material is formed into a the shape of a bullet, having a tapered end. The earplug can be inserted deep inside the ear canal and consequently has a good soundproofing effect. Even though it is inserted deep inside the ear canal, it can be removed easily by grasping the removal extension at the end of the plug opposite to the tapered end. The film avoids adherence of ear wax and other kinds of dirt to the plug, allowing the plug to be used for a long time.

### BRIEF DESCRIPTION OF DRAWINGS

FIGS. 1(a) and 1(b) are respectively a schematic view and an exploded view of the earplug according to the invention;

FIG. 2 is a schematic view illustrating a method of insertion of the earplug according to the invention;

FIGS. 3(a) and 3(b) are respectively an exterior view and a cross-sectional view showing the earplug of the invention inserted in a user's ear;

FIGS. 4(a) and 4(b) are respectively a view showing a conventional sponge earplug held prior to insertion, and a cross-sectional view showing the same sponge earplug inserted in a user's ear; and

FIGS. 5(a) and 5(b) are respectively a view showing a conventional silicone earplug in a generally spherical condition prior to insertion, and a cross-sectional view showing the same silicone earplug inserted in a user's ear.

### DETAILED DESCRIPTION OF THE PREFERRED EMBODIMENTS

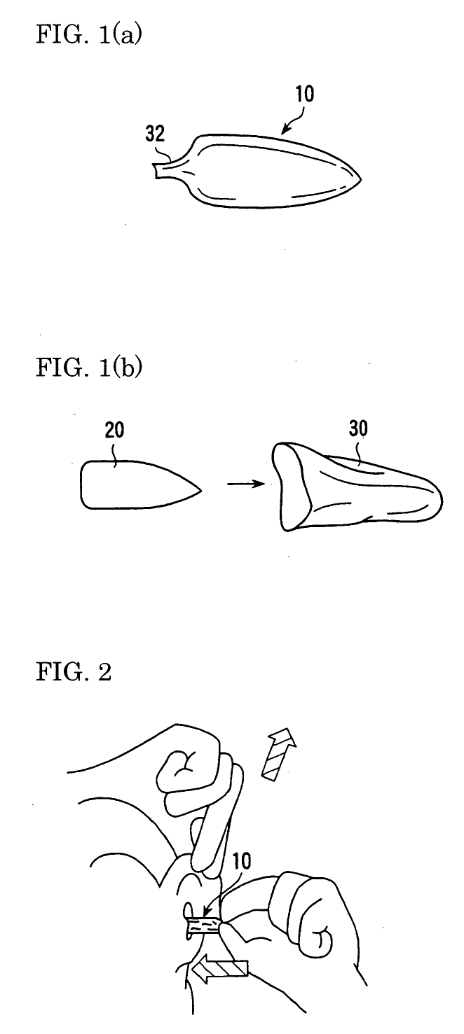

FIG. 1(a) shows an earplug 10 according to the invention. As shown in FIG. 1(b) the earplug 10 comprises a main plug body 20, and a plastic film cover 30 that encloses the plug body 20, preferably closely fitting the plug body in such a way as to avoid significant air pockets. The plug body 20 is preferably formed in the shape of a bullet, having a tapered end. The point of the tapered end can face the entrance of the ear canal at the time of insertion of the plug. The plug body comprises a material that has is substantially non-resilient, and exhibits plasticity at least at the temperatures to which the earplug will ordinarily be subjected, i.e., temperatures in the range from an ambient temperature of about 20 EC to body temperature (about 37 EC), so that the earplug can be easily deformed manually. The size of the ear canal varies from one person to another, but the user can decide how narrow the plug should be in order to fit his or her ear canal, and shape the plug accordingly while it is inside the plastic film cover 30, as shown in FIG. 1(a).

When the plug body 20 is within the film cover 30 a part 32 of the cover extends from the rear end of the plug body in a direction opposite to the direction in which the tapered end of the plug body points. This part of the cover serves as a manually graspable removal extension, by which the ear plug can be removed readily from the ear canal.

Because it is covered by a plastic film, the direct adherence of ear wax or other kinds of dirt to the plug body 20 is avoided. Accordingly the ear plug can be used for a longer time than a conventional ear plug. Moreover, because the plug body is enclosed within the film cover, and the film cover has a removal extension 32, even if the plug body is broken while deep inside the ear canal, the entire plug can be removed easily. Furthermore, since this earplug 10 is substantially non-resilient, it does not give users an uncomfortable oppressive feeling as in the case of the sponge-type earplug, and can be kept inside one's ear comfortably for a long time.

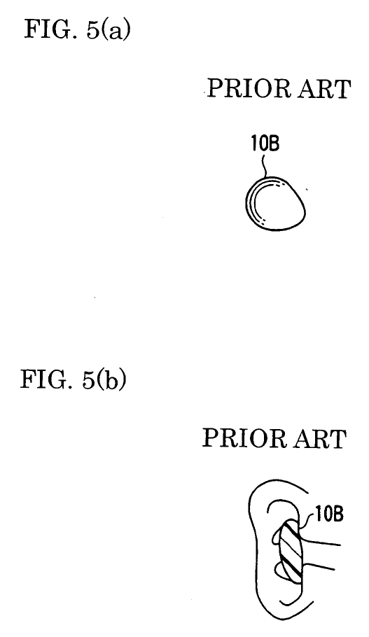

The plug body 20 preferably comprises a low-resilience silicone that has sufficient plasticity at least at ordinary temperatures, i.e. temperatures in the range from about 20 EC to about 37 EC, that it can be manually deformed, but hold its shape, when deformed. The plug body 20 can be composed of the same silicone material as that of a conventional silicone ear plug of the type shown in FIG. 5.

As an alternative, especially since the plug body 20 is encased in a film cover, other materials besides silicone can be used to form the plug body. For example, materials such as petroleum jelly (e.g. the petroleum jelly produced and sold under the trademark VASELINE, by Chesebrough-Ponds, Inc. of Greenwich, Conn.), or cotton impregnated with petroleum jelly, can be used instead of silicone. For the cover 30, a plastic film of polyethylene or poly (vinyl chloride), is preferred. The plastic film must be sufficiently thin that it will not impair the plasticity of the plug body therein, but must have enough strength that it cannot be broken easily when pulled. The removal extension 32 may be conveniently formed by sealing the end of the cover 30 located adjacent the end of the plug body opposite to the tapered end. As shown in FIG. 1(b), preferably the cover 30 is initially open at this end in order to receive the plug body 20. Conventional sealing techniques, such as heat sealing or adhesion by an adhesive agent can be used.

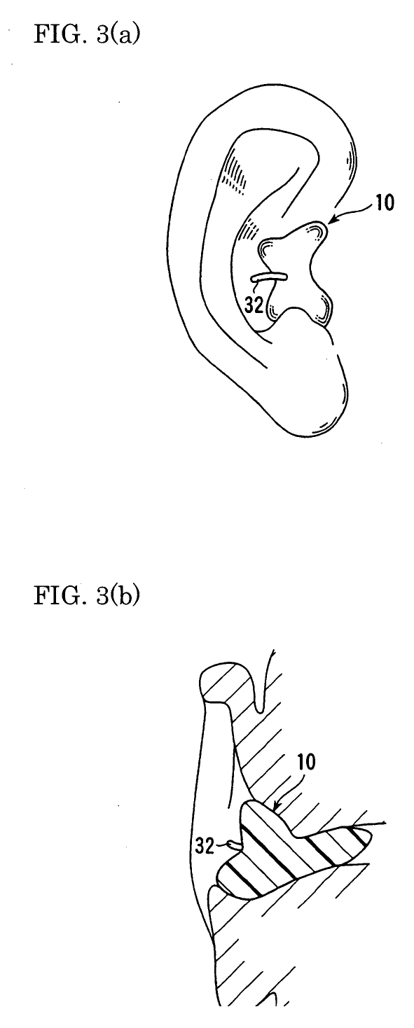

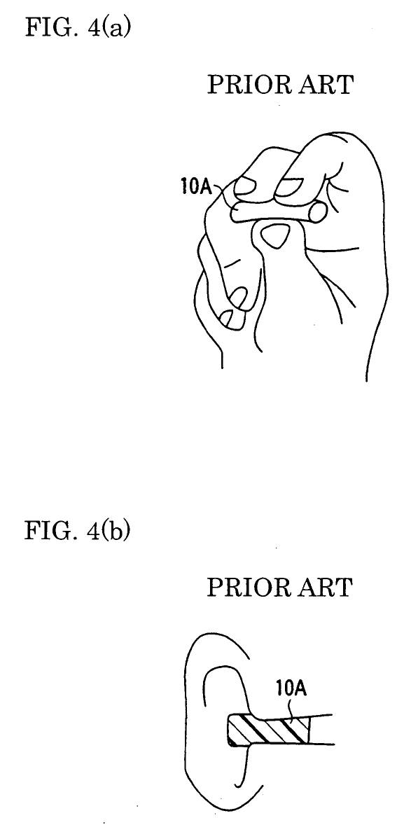

The method of insertion of the earplug according to the invention is illustrated in FIG. 2. When inserting the earplug into one's left ear, as in the case of a conventional earplug, the right hand is extended behind the user's head to grasp and pull rearwardly on the left ear, thereby straightening the ear canal. With the other hand, the user can easily insert the earplug, and push into the ear canal so that the tip of the plug is positioned well inside the ear canal. If the earplug is too large to fit into the user's ear canal, it may be deformed into a narrower shape. When the ear is released, the outer part of the plug adheres to the ear at the entrance of the ear canal. After insertion, the ear plug can be further deformed by pressing on its exposed end, to block the entrance of the ear canal completely as shown in FIGS. 3(a) and 3(b). As shown in these two figures, the earplug can achieve a waterproofing effect by spreading over the entrance of the ear canal, thereby completely blocking the passage of water into the ear. As seen in FIGS. 3(a) and 3(b) the removal extension 32 is exposed at a location at which it can be grasped easily for removal of the entire earplug from the ear.

According to the invention, with the plug body, which comprising a material that exhibits plasticity at ordinary temperatures being wholly covered by a plastic film cover, the earplug can be deformed into a narrow bullet shape and can be inserted inside one's ear canal. Therefore, a superior soundproofing effect can be obtained. In addition, even though the earplug can be pushed deep inside the ear canal, it can be removed easily by grasping the removal extension, which is a part of the cover film exposed toward the outside of the ear canal while the earplug is in place. Moreover, because the plug body is entirely covered by the plastic film cover, no adhesion of ear wax or other material onto the plug body occurs, and therefore the plug can be kept clean and used for a long time.

1-12. (canceled)

13. An earplug comprising a plug of material that exhibits sufficient plasticity at ordinary temperatures that it can be deformed manually, and a flexible film cover enclosing said plug of material, wherein said flexible film cover is initially in the shape of a bag having an entrance at one end thereof through which said plug of material can be inserted, whereby the film cover closely fits the plug, wherein a portion of said flexible film cover extends away from the enclosed material, forming an extension that can be grasped manually for removal of the earplug from the ear, and wherein said flexible film cover fits the plug sufficiently closely that the interior of the film cover is substantially free of air pockets.

14. An earplug comprising a plug of material that exhibits sufficient plasticity at ordinary temperatures that it can be deformed manually, and a flexible film cover enclosing said plug of material, wherein said flexible film cover is initially in the shape of a bag having an entrance at one end thereof through which said plug of material can be inserted, whereby the film cover closely fits the plug, wherein a portion of said flexible film cover extends away from the enclosed material, forming an extension that can be grasped manually for removal of the earplug from the ear, and wherein said flexible film cover is tubular in shape, having a side wall surrounding the plug of material, and an end wall at one end, the end wall being continuous with the side wall, and the total area of said flexible film cover being greater than the area of said entrance when said entrance is fully open.

15. (cancelled)
